(19) United States
(12) Patent Application Publication
(10) Pub. No.: US 2025/0032619 A1
(43) Pub. Date: Jan. 30, 2025

(54) MICROFLUIDIC PREPARATION OF FLUOROCARBON CROSS-LINKED VESICLES

(71) Applicant: Bracco Suisse SA, Cadempino (CH)

(72) Inventors: Raphaël DE MATOS, Lausanne (CH); Romain MELICH, Choisy (FR); Anthony CLOUET, Prevessin-Moens (FR); Samir CHERKAOUI, Feigeres (FR)

(73) Assignee: Bracco Suisse SA, Cadempino (CH)

(21) Appl. No.: 18/708,660

(22) PCT Filed: Nov. 11, 2022
(86) PCT No.: PCT/EP2022/081666

§ 371 Date: May 9, 2024

(30) Foreign Application Priority Data
Nov. 11, 2021 (EP) 21207707.7

(51) Int. Cl. A61K 41/00; A61K 9/00; A61K 49/22

(52) U.S. Cl. CPC A61K 41/0028; A61K 9/0009; A61K 49/223; A61K 2123/00

(57) ABSTRACT

The present invention relates to a method for the preparation of calibrated (per)fluorocarbon cross-linked vesicles through microfluidic technique. The invention further relates to an aqueous suspension comprising a plurality of calibrated fluorocarbon cross-linked vesicles obtained by said method for use in a diagnostic and/or therapeutic treatment.

### SEQUENCE LISTING

The instant application contains a Sequence Listing which has been submitted electronically in XML file format and is hereby incorporated by reference in its entirety. Said XML copy, created on May 28, 2024, is named 01122_0111_SL.xml and is 7,693 bytes in size.

### TECHNICAL FIELD

The invention generally relates to a method of preparation of an aqueous suspension of calibrated fluorocarbon cross-linked vesicles through a microfluidic technique. The invention further relates to an aqueous suspension comprising a plurality of calibrated fluorocarbon cross-linked vesicles obtained by said method for use in a diagnostic and/or therapeutic treatment.

### BACKGROUND OF THE INVENTION

Phase-change contrast agents (PCCAs) or acoustically activated nanodroplets are receiving increased popularity in both ultrasounds diagnostic and therapeutic delivery. Except for the core, often consisting of liquid perfluorocarbons, nanodroplets display similar composition to commercially available gas-filled microbubbles. Owing to Acoustic Droplet Vaporization (ADV) process, encAPulated droplets are converted into gas bubbles upon exposure to ultrasound energy beyond a vaporization threshold. In fact, ultrasounds act as a remote trigger to promote the vaporization of the droplets in a controllable, non-invasive and localized manner. Thanks to their smaller size compared to conventional microbubbles, nanodroplets display prolonged in vivo circulation and deep penetration into the tissues via the extravascular space. Moreover, below the vaporization threshold, they are ultrasonically stable with low acoustic attenuation and can be acoustically vaporized at the location of interest.

Perfluorocarbon nanodroplets (“PFC-NDs”) present a real potential as an extravascular ultrasound contrast agent in numerous diagnostic and therapeutic applications including sonopermeabilization, thermal ablation, blood-brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging since these agents can be activated and deactivated on demand by applying intermittent acoustic pulses.

A major limitation of nanodroplets is their relatively limited physico-chemical stability over time, which may affect their use in diagnostic and therapeutic applications.

Melich et al, 2020 report the use of rapid and controlled microfluidic mixing for the manufacturing of various types of PFC-NDs with different stabilizing shells.

WO2019/023706 discloses PFC-NDs (i.e. peptisomes) having a perfluorocarbon liquid core containing a cargo, e.g. a therapeutically active agent, and a plurality of cross-linkable amphiphilic peptide molecules comprising a fluorinated hydrophobic block, such as a fluorinated hydrophobic amino acid sequence, a cross-linking motif, and a hydrophilic amino acid sequence. Peptisomes are obtained through a solvent-exchange procedure in which water is slowly added to an organic emulsion of peptide and PFC, ultimately leading to the spontaneous assembly of the peptide at the surface of PFC nanodroplets.

Up to now, according to Applicants' knowledge, such PFC-NDs stabilized by said cross-linkable amphiphilic peptides have not been prepared yet through microfluidic techniques.

The Applicants have now developed a microfluidic method for the preparation of a composition comprising calibrated (per) fluorocarbon cross-linked vesicles, stabilized by amphiphilic peptides.

Generally in the state of the art, the term “calibrated” is also indicated as “size-controlled”, “uniform-sized droplets”, “monodisperse (d)” or “monosize (d)”.

### SUMMARY OF THE INVENTION

An aspect of the invention relates to a method for the preparation of an aqueous suspension of calibrated fluorocarbon cross-linked vesicles, said method comprising the steps of:
a) Preparing an aqueous phase wherein the temperature of said aqueous phase is at least of 10° C.;
b) Preparing an organic phase, wherein
   i) said aqueous phase comprises a cross-linkable amphiphilic peptide and said organic phase comprises a fluorocarbon or
   ii) said organic phase comprises a cross-linkable amphiphilic peptide and a fluorocarbon;
c) Injecting said aqueous phase in a first inlet and said organic phase in a second inlet of a microfluidic cartridge, thereby mixing said aqueous phase and said organic phase in a mixing portion of the microfluidic cartridge to obtain an aqueous suspension of calibrated fluorocarbon cross-linkable vesicles;
d) Collecting the aqueous suspension of calibrated fluorocarbon cross-linkable vesicles from an exit channel of the microfluidic cartridge, and
e) Cross-linking the cross-linkable amphiphilic peptides to obtain an aqueous suspension of calibrated fluorocarbon cross-linked vesicles.

In a preferred embodiment, at step a) the temperature of said aqueous phase is at least 15° C., more preferably at least of 18° C., still more preferably at least of 19° C. The temperature of the aqueous phase is typically not higher than the boiling point of the components of said organic phase, preferably not higher than the boiling point of the component of the organic phase having the lowest boiling point. For example the temperature of the aqueous phase is lower than 60° C., more preferably lower than 30° C., still more preferably lower than 25° C., preferably lower than 22° C., more preferably the temperature is at least 20° C.

Preferably the temperature is at least 20° C.±1 (i.e. room temperature).

Preferably said aqueous phase comprises a cross-linkable amphiphilic peptide and the organic phase comprises a fluorocarbon.

Preferably said fluorocarbon is a perfluorocarbon.

In a further embodiment, said aqueous phase comprises an aqueous liquid component having a pH lower than 7.0, more preferably lower than 6.5, still more preferably lower than 6.0, more preferably lower than 5.5., more preferably lower than 5, more preferably lower than 4.5, still more preferably lower than 4.0, up to e.g. 0, preferably up to 1.0.

In an embodiment, said aqueous phase comprises an aqueous component selected from water, sodium acetate buffer, tris(hydroxymethyl)aminomethane buffer or a mixture thereof.

In another embodiment, said organic phase comprises an organic solvent selected from ethanol, methanol or a mixture thereof.

In an embodiment, said cross-linkable amphiphilic peptide is a compound of formula I:

HB-CL-HP (I)
wherein HB is a fluorinated hydrophobic polymer, CL is a cross-linking motif and HP is a hydrophilic amino acid sequence.

In a preferred embodiment, the cross-linkable amphiphilic peptide is a compound of Formula (II):

HB′-CL-HP (II)

wherein HB′ is a fluorinated hydrophobic amino acid sequence; CL is a cross-linking motif; and HP is a hydrophilic amino acid sequence.

In a further preferred embodiment, the cross-linkable amphiphilic peptide is the compound of Formula IV (SEQ ID NO: 4):

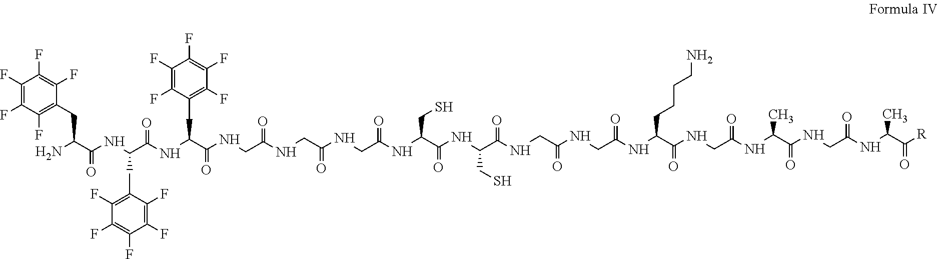

Wherein the hydrophobic portion is pentafluorophenylalanine, the cross-linking motif is GGGCCGG (SEQ ID NO: 1), the hydrophilic peptide is the amino acid sequence Alanine Glycine Alanine (i.e. AGA) and R is —OH or NH₂.

When R is —OH the compound of formula IV is referred to as F_{f}F_{f}F_{f}GGGCCGGKGAGA (SEQ ID NO:2).

When R is —NH₂ the compound of formula IV is referred to as F_{f}F_{f}F_{f}GGGCCGGKGAGA-NH₂ (SEQ ID NO: 3).

In an embodiment, the method further comprises an optional step d′), which comprises diluting the aqueous suspension of calibrated fluorocarbon cross-linkable vesicles.

In another embodiment the method further comprises an optional step f), which comprises washing the obtained aqueous suspension of calibrated fluorocarbon cross-linked vesicles.

Another aspect of the invention relates to an aqueous suspension comprising a plurality of calibrated fluorocarbon cross-linked vesicles obtained according to the process as above defined, wherein said calibrated (per) fluorocarbon cross-linked vesicles have a z-average diameter comprised between 100 nm and 1000 nm and a polydispersity lower than 0.25.

In a preferred embodiment, said cross-linked vesicles have a degree of cross-linking higher than 80%, preferably at least 85%, more preferably at least 90%, still more preferably at least 95%. Preferably the degree of cross-linking is 100%.

In a further embodiment said aqueous suspension comprising a plurality of calibrated fluorocarbon cross-linked vesicles further comprises a pharmaceutically acceptable excipient.

In a preferred embodiment said pharmaceutically acceptable excipient is glucose.

A further aspect relates to an aqueous suspension comprising a plurality of calibrated fluorocarbon cross-linked vesicles obtained according to the process as above defined for use in a diagnostic and/or therapeutic treatment.

### FIGURES

FIG. 1 is a schematic representation of the core portion of a microfluidic cartridge.

FIG. 2 shows a schematic representation of a cross-section of a staggered herringbone mixer (SHM) design inside the channel.

### DETAILED DESCRIPTION OF THE INVENTION

The present invention generally relates to a microfluidic method for the preparation of a composition comprising calibrated (per) fluorocarbon cross-linked vesicles, stabilized by cross-linkable amphiphilic peptides and to the vesicles obtained by said method.

Said calibrated cross-linked vesicles are suitable as contrast agents in ultrasound imaging techniques, known as Contrast-Enhanced Ultrasound (CEUS) Imaging, in molecular imaging to target specific receptors, or in therapeutic applications, e.g. thermal ablation or for ultrasound-mediated drug/gene delivery.

### Definitions

The term “vesicle” indicates an assembly comprising an outer layer and an inner core, said outer layer comprising a cross-linkable amphiphilic peptide and said inner core comprising a (per) fluorocarbon. In said vesicles, the cross-linkable amphiphilic peptide molecules are oriented in such a way that the hydrophobic portions of the peptide are located at a surface of the (per) fluorocarbon of the inner core.

The expression “cross-linkable amphiphilic peptide” refers to any amphiphilic peptide comprising cross-linkable moieties that can potentially be covalently linked to each other through a cross-linking reaction. Suitable examples of cross-linkable moieties are cross-linkable aminoacids such as cysteine residues, that can be intermolecularly connected to an adjacent cysteine residue via disulfide cross-linking groups (—S—S—).

In a preferred embodiment, said cross-linkable moieties are cross-linkable amino acids, preferred being cysteine amino acids comprised in adjacent cross-linkable amphiphilic peptides.

In the present description and claims, the expression “cross-linking reaction” indicates the process of forming covalent bonds between cross-linkable moieties comprised in adjacent cross-linkable amphiphilic peptides in order to bind cross-linkable amphiphilic peptides molecules together.

The term “cross-linkable vesicle” indicates an assembly comprising an outer layer and an inner core, said outer layer comprising a cross-linkable amphiphilic peptide and said inner core comprising a (per) fluorocarbon, wherein said cross-linkable amphiphilic peptide is not bonded intermolecularly to an adjacent amphiphilic peptide. As stated above, the cross- linkable amphiphilic peptides forming the outer layer of the cross-linkable vesicles comprise cross-linkable moieties that can potentially be covalently linked to each other.

The term “cross-linked vesicle” indicates an assembly comprising an outer layer and an inner core, said outer layer comprising a cross-linkable amphiphilic peptide and said inner core comprising a (per) fluorocarbon, wherein said cross-linkable amphiphilic peptide is covalently linked to an adjacent cross-linkable amphiphilic peptide through the cross-linking of the cross-linkable moieties. For instance, when the cross-linkable amphiphilic peptide includes a cysteine residue, said cysteine residue may be intermolecularly connected to an adjacent cysteine residue via disulfide cross-linking groups (—S—S—).

Preferably the vesicles of the present invention are perfluorocarbon nanodroplets, wherein the term nanodroplets refers to vesicles having a z-average diameter comprised between 100 nm and 1000 nm.

The expression “calibrated” refers to a population of (per) fluorocarbon vesicles as above defined (either cross-linkable or cross-linked), wherein said vesicles have a z-average diameter comprised between 100 nm and 1000 nm and a polydispersity lower than 0.25.

In the present invention said calibrated (per) fluorocarbon vesicles are preferably obtained through microfluidic technique.

The expression “calibrated (per) fluorocarbon cross-linkable vesicles” indicates an aqueous suspension comprising a plurality of calibrated (per) fluorocarbon cross-linkable vesicles as above defined, preferably obtained through microfluidic technique. A suspension of calibrated (per) fluorocarbon cross-linkable vesicles has not been yet submitted to any procedure aiming at inducing the cross-linking of the cross-linkable amphiphilic peptides forming the outer layer of the vesicles.

The expression “calibrated (per) fluorocarbon cross-linked vesicles” indicates an aqueous suspension of calibrated (per) fluorocarbon cross-linked vesicles as above defined, said suspension being obtained through microfluidic technique. After the collection from the microfluidic cartridge, e.g. within 5 minutes, an aqueous suspension of calibrated (per) fluorocarbon cross-linkable vesicles can be submitted to a procedure aiming at inducing the cross-linking of the cross-linkable amphiphilic peptides forming the outer layer of the vesicles. As result, an aqueous suspension of calibrated (per) fluorocarbon cross-linked vesicles is obtained.

### Cross-Linkable Amphiphilic Peptide

The expression “cross-linkable amphiphilic peptide” (“CAP”) refers to a peptide of formula I

HB-CL-HP (I)
wherein HB is a fluorinated hydrophobic polymer, CL is a cross-linking motif and HP is a hydrophilic amino acid sequence.

The cross-linkable amphiphilic peptide has a molecular weight in the range of about 1000-5000 daltons, wherein the cross-linkable amphiphilic peptide comprises from 5 to 50 amino acids residues, preferably from 5 to 40, more preferably from 5 to 30, wherein at least two of the amino acid residues are consecutively linked to each other in a chain by a peptide bond.

Cross-linkable amphiphilic peptides can be synthesized using techniques known to one of ordinary skill in the art, such as, but not limited to, solid-phase synthesis, recombinant methodologies polymerization, and conjugation methods.

As used herein, the term “fluorinated hydrophobic polymers” refers to a covalently linked chain of monomer residues forming a fluorinated hydrophobic homopolymer or copolymer. The monomeric units which form the fluorinated hydrophobic polymer may each be fluorinated according to embodiments, or some, or one, of the monomeric units is fluorinated such that at least one or more of the monomer residues of the fluorinated hydrophobic polymer is fluorinated.

According to an embodiment, the cross-linkable amphiphilic peptide does not include lipids.

According to another embodiment, the fluorinated hydrophobic polymer includes a hydrophobic amino acid sequence wherein the amino acids of the hydrophobic amino acid sequence have non-polar side chains, wherein the non-polar side chains do not include a group capable of forming a hydrogen bond with molecules of water, and wherein at least one of the amino acids of the hydrophobic amino acid sequence is fluorinated.

According to an embodiment, the fluorinated hydrophobic polymer includes one or more synthetic non-amino acid monomeric units wherein at least one of the monomeric units is fluorinated such that at least one of the monomer residues of the fluorinated hydrophobic polymer is fluorinated. Non limiting examples of synthetic monomeric units which can be fluorinated and reacted to form a fluorinated hydrophobic polymer include methyl methacrylate, lactic acid, glycolic acid and olefins such as ethylene, propylene, styrene.

In one embodiment, the cross-linkable amphiphilic peptide is a compound of Formula (II):

HB′-CL-HP (II)

wherein HB′ is a fluorinated hydrophobic amino acid sequence; CL is a cross-linking motif; and HP is a hydrophilic amino acid sequence.

In another embodiment, the cross-linkable amphiphilic peptide is a compound of Formula (III):

HB′-CL-HP —NH₂ (III)

wherein HB′ is a fluorinated hydrophobic amino acid sequence; CL is a cross-linking motif; and HP is a hydrophilic amino acid sequence wherein the C-terminal amino acid is amidated.

As used herein, the term “hydrophobic amino acid sequence” refers to a sequence of hydrophobic amino acids having non-polar side chains, wherein the non-polar side chains do not include a group capable of forming a hydrogen bond with molecules of water, or a combination of a hydrophobic polymer and a sequence of hydrophobic amino acids having non-polar side chains. Hydrophobic amino acids may be naturally occurring or non-natural (artificially produced). Examples of the naturally occurring hydrophobic amino acids include, but are not limited to, alanine, valine, leucine, isoleucine, proline, phenylalanine, tryptophan, cysteine, and methionine. Examples of non-natural hydrophobic amino acids may include D amino acids, as well as specific non-natural amino acids such as selenocysteine, pyrrolysine, and the like.

In the cross-linkable amphiphilic peptide, the fluorinated hydrophobic amino acid sequence may include one to ten fluorinated hydrophobic amino acids consecutively connected by peptide bonds, which may be unsubstituted or substituted with a substituent selected from —F, —CI, —Br, —I, a C₁-C₃₀ alkyl group, a C₂-C₃₀ alkenyl group, a C₂-C₃₀ alkynyl group, a C₃-C₃₀ cycloalkyl group, a C₃-C₃₀ cycloalkenyl group, a C₆-C₃₀ aryl group, a C₇-C₃₀ arylalkyl group, but are not limited thereto. Fluorinated hydrophobic amino acids include, for example, fluorinated alanine, fluorinated valine, fluorinated leucine, fluorinated isoleucine, fluorinated proline, fluorinated phenylalanine, fluorinated tryptophan, fluorinated cysteine, fluorinated methionine, fluorinated selenocysteine and fluorinated pyrrolysine. The fluorinated hydrophobic amino acids can be D or L amino acids and can be fluorinated at any suitable position, typically replacing a hydrogen atom.

In a preferred embodiment, the fluorinated hydrophobic amino acids sequence may include pentafluoro-phenylalanine (2,3,4,5,6-pentafluoro-L-phenylalanine and/or 2,3,4,5,6-pentafluoro-D-phenylalanine) at a terminal thereof.

In another embodiment, the fluorinated hydrophobic amino acid sequence may comprise one to ten consecutively connected pentafluoro-phenylalanine residues at a terminal thereof.

Preferably, the fluorinated hydrophobic amino acid sequence comprises three consecutively connected pentafluoro-phenylalanine residues at a terminal thereof.

Suitable fluorinated amino acids are those described for instance in WO 2019/023707.

A combination of a hydrophobic polymer and a sequence of hydrophobic amino acids having non-polar side chains can be included in the fluorinated hydrophobic polymer wherein at least one of the monomer residues of the fluorinated hydrophobic polymer is fluorinated and/or at least one of the amino acid residues is fluorinated.

As used herein, the term “hydrophilic amino acid sequence” refers to a sequence of hydrophilic amino acids consecutively connected by peptide bonds, wherein the hydrophilic amino acids have a polar side chain, wherein the polar side chain includes a group capable of forming a hydrogen bond with molecules of water. Hydrophilic amino acids may be naturally occurring or non-natural and can be D or L amino acids. Examples of the naturally occurring hydrophilic amino acids include, but are not limited to, serine, threonine, asparagine, glutamine, histidine and tyrosine.

Examples of non-natural hydrophilic amino acids include amino acids having various heterocyclic groups as a part of the side chain.

In the cross-linkable amphiphilic peptide, the hydrophilic amino acid sequence HP may include from 3 to 40 hydrophilic amino acids, preferably from 3 to 30, more preferably from 3 to 20, consecutively connected by peptide bonds.

In another embodiment, the hydrophilic amino acid sequence comprises a targeting agent that interacts (e.g. through covalent or non-covalent binding) with a specific moiety, such as a receptor expressed by a target cell, resulting in the binding of the vesicle to said moiety or molecule.

The target cells can be cells of any organism, such as, but not limited to, a mammal, bird, fish, or bacterial cell. According to an embodiment, the target cell is a human cell or a bacterial cell within a human body.

The targeting agent may include a minimal targeting motif peptide and optionally includes one or more hydrophilic amino acids attached to the N-terminus or C-terminus of the minimal targeting motif peptide by peptide bonds.

Typically, amino acids of the targeting motif peptide are L-amino acids but these may include one or more D-amino acids so long as the targeting motif still correctly mediates binding with the receptor. The one or more hydrophilic amino acids attached to the N-terminus or C-terminus of the minimal targeting motif peptide by peptide bonds can be D or L amino acids.

Examples of suitable targeting motifs and examples of hydrophilic amino acid sequences including a targeting motif that can be comprised in the cross-linkable amphiphilic peptides are described, for instance, in WO 2019/023707.

In the present description and claims, the expression “cross-linking motif” refers to a cross-linkable moiety, comprised in the cross-linkable amphiphilic peptide, that can potentially be covalently linked to another cross-linkable moiety through a cross-linking reaction. Examples of cross-linking motif are sulfhydryl cross-linkers, UV cross-linkers, aza-benzenes, photosensitive cross-linkers, such as azides or benzophenones, nitriles, pH-sensitive cross-linkers, or enzymatic cross-linkers and click-chemistry-based cross-linkers (e.g. a cross-linking motif comprising an azide group able to react with an alkyne-containing cross-linking motif via click chemistry reactions, such as the copper-catalyzed azide alkyne cycloaddition).

In a preferred embodiment, the “cross-linking motif” is an amino acids sequence comprising at least one cross-linkable amino acid residues that can potentially be covalently linked to corresponding cross-linkable amino acid residues through a cross-linking reaction. The cross-linking motif may comprise from 1 to about 40 amino acid residues, preferably from 3 to 30, more preferably from 3 to 20.

In the cross-linking motif said cross-linkable amino acid residues may be at any position in the sequence and can be naturally occurring amino acids and/or non-naturally occurring amino acids.

An example of a naturally occurring amino acid able to cross-link with a corresponding cross-linkable amino acid residue is cysteine.

Non-naturally occurring amino acids may be obtained through structure functionalization of naturally occurring amino acids providing the ability to bind to a naturally occurring or non-naturally occurring amino acid in the crosslinking motif of an adjacent cross-linkable residue.

In an embodiment, the cross-linking motif comprises a cysteine.

In a further embodiment, the cross-linking motif comprises a cysteine and a glycine.

In a preferred embodiment, the cross-linking motif comprises the amino acid sequence GGGCCGG (SEQ ID NO: 1), wherein G is glycine and C is cysteine.

In an embodiment, the degree of cross-linking of the cross-linkable amphiphilic peptide molecules is higher than 80%, preferably at least 85%, more preferably at least 90%, still more preferably at least 95%. Preferably the degree of cross-linking is 100%.

The expression “degree of cross-linking” refers to the total number of cross-linked amino acid residues, e.g. cysteines, that interconnect adjacent cross-linkable amphiphilic peptides. The degree of cross-linking is generally expressed in percent and can be measured using a colorimetric disulfide formation assay.

In the present invention, preferred are cross-linkable amphiphilic peptides comprising three pentafluoro-phenylalanine (F_{f}) residues at the N-terminus. C-terminal to this fluorinated domain is a cysteine containing crosslinking motif (GGGCCGG, SEQ ID NO: 1).

In a preferred embodiment, the cross-linkable amphiphilic peptide is the compound of formula IV (SEQ ID NO: 4)

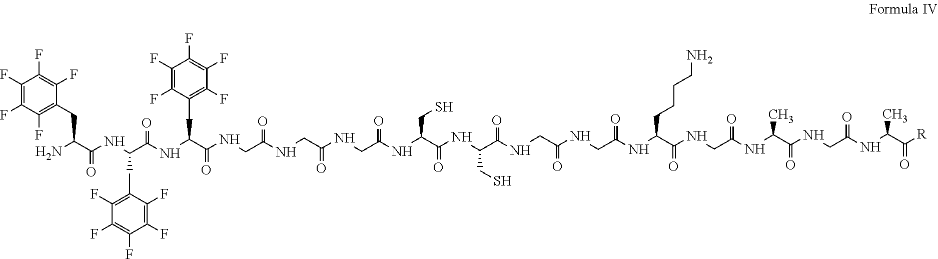

Wherein the hydrophobic portion is pentafluorophenylalanine, the cross-linking motif is GGGCCGG (SEQ ID NO: 1), the hydrophilic peptide is the amino acid sequence Alanine Glycine Alanine (i.e. AGA) and R is —OH or NH₂.

When R is —OH the compound of formula IV is referred to as F_{f}F_{f}F_{f}GGGCCGGKGAGA (SEQ ID NO:2).

When R is —NH₂ the compound of formula IV is referred to as F_{f}F_{f}F_{f}GGGCCGGKGAGA-NH₂ (SEQ ID NO:3).

### (Per)fluorocarbons

As described above, “cross-linked vesicle” indicates an assembly comprising an outer layer and an inner core, said outer layer comprising a cross-linkable amphiphilic peptide and said inner core comprising a (per) fluorocarbon that allows for activation of the vesicle upon application of ultrasound (US).

In the present description and claims, the term “fluorocarbons” refers to a group of fluorine-containing compounds derived from hydrocarbons by partial or complete substitution of hydrogen atoms with fluorine atoms, which are liquid at room temperature. Preferably the fluorocarbon is a perfluorocarbon (PFC), i.e. a fluorinated hydrocarbon where all the hydrogen atoms are substituted with fluorine atoms.

Liquid (per) fluorocarbons are characterized by a boiling point comprised between 25° C. and 160° C. In the present invention, the (per) fluorocarbons are preferably characterized by a boiling point comprised between 25° C. and 100° C., still more preferably between 27° C. and 60° C. Suitable examples of fluorocarbons are 1 1-Fluorobutane, 2-Fluorobutane, 2,2-Difluorobutane, 2,2,3,3-Tetrafluorobutane, 1,1,1,3,3-Pentafluorobutane, 1,1,1,4,4,4-Hexafluorobutane, 1,1,1,2,4,4,4-Heptafluorobutane, 1,1,2,2,3,3,4,4-Octafluorobutane, 1,1,1,2,2-Pentafluoropentane, 1,1,1,2,2,3,3,4-Octafluoropentane, 1,1,1,2,2,3,4,5,5,5-Decafluoropentane, 1,1,2,2,3,3,4,4,5,5,6,6-Dodecafluorohexane.

Suitable examples of perfluorocarbons are perfluoropentane, perfluorohexane, perlfluoroheptane, perfluorooctane, perfluorononane, perfluorodecalin, perfluorooctylbromide (PFOB), perfluoro-15-crown-5-ether (PFCE), perfluorodichlorooctane (PFDCO), perfluorotributylamine (PFTBA), perfluorononane (PFN), and 1,1,1-tris (perfluorotert-butoxymethyl) ethane (TPFBME), or a mixture thereof.

In an embodiment said perfluorocarbon is preferably perfluoropentane (PFP) (boiling point 29° C.) or perfluorohexane (PFH) (boiling point 57° C.).

### Drug/Gene Delivery

The expression “drug/gene delivery” indicates a therapeutic protocol comprising the administration of at least a bioactive agent, said bioactive agent being comprised in the cross-linked vesicles.

Bioactive agent includes any molecule, compound, formulation or material capable of producing a biologically or therapeutically active effect on the region or organ to be treated. Said bioactive agent can be comprised in the cross-linked vesicles through different mechanisms, such as i) by incorporation within the inner core of the cross-linked vesicles or ii) by covalent/electrostatic interaction with the outer shell.

In an embodiment, the bioactive agent can be incorporated within the inner core for instance through its dispersion in the perfluorocarbon liquid core.

In another embodiment, the bioactive agent can be contacted with a fluorine solubilizing vehicle containing the bioactive agent to aid in miscibility with the perfluorocarbon core. Examples of fluorine solubilizing vehicles are perfluoroalkyls, polyfluoroalkyls, perfluorinated alkyl acids, polyfluorinated alkyl acids, perfluorinated aromatic compounds, polyfluorinated aromatic compounds, any of which may be further substituted or unsubstituted, or a mixture of any two or more thereof.

Alternatively, the bioactive agent can be comprised on the cross-linked vesicles through the binding with the outer shell (i.e. with the cross-linkable amphiphilic peptides). For example, said binding can be based on different physico-chemical interactions, such as covalent and/or electrostatic.

In order to covalently or electrostatically bind a bioactive agent to the outer shell of the cross-linked vesicles, specific reactive moiety need to be present on the cross-linkable amphiphilic peptides structure. For example, the cross-linkable amphiphilic peptide may comprise suitable amino acid able to give an overall charge to the cross-linked vesicles in order to electrostatically bind a bioactive agent owing an opposite charge.

Suitable examples of bioactive agents include therapeutically active agents, for example a small molecule therapeutic agent, a protein therapeutic agent, a peptide therapeutic agent, a nucleic acid-based agent, such as RNA, DNA, an miRNA molecule, an siRNA molecule, an shRNA molecule, a dsRNA molecule, an antisense molecule, a ribozyme, a polynucleotide encoding an miRNA, siRNA, shRNA, dsRNA, or a combination, of any two or more thereof, a gene editing tool, but is not limited thereto.

The therapeutically active agent may further include a radioisotope, an antithrombotic agent, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

### Microfluidic Cartridge

As mentioned above, the calibrated (per) fluorocarbon cross-linked vesicles according to the invention are prepared by using a microfluidic technique.

In the present description and claims, the expression “microfluidic technique” refers to a technology of manufacturing cross-linked vesicles through a microfluidic cartridge designed to manipulate fluids in channels at the microscale.

Said microfluidic technique is a bottom up approach, that is to say, that the cross-linked vesicles are obtained by assembling molecules (e.g. cAP and (per) fluorocarbons) into larger structures (i.e. cross-linkable and cross-linked vesicles).

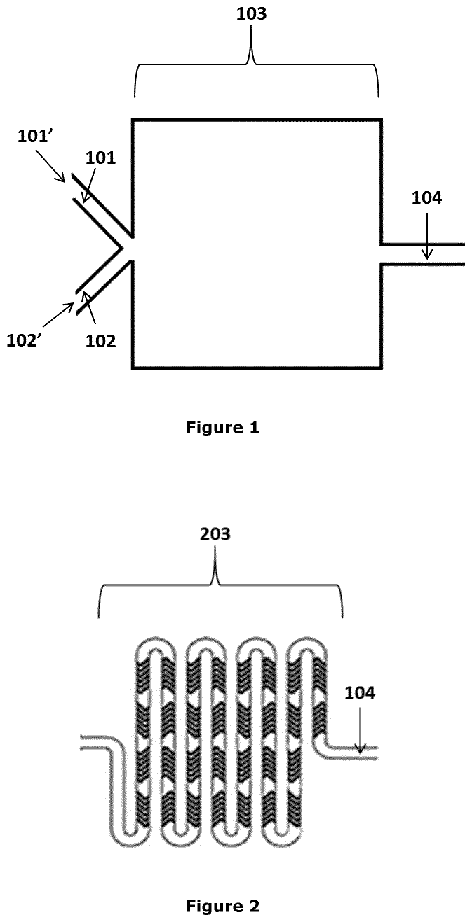

FIG. 1 shows a schematic representation of the core portion 100 of a microfluidic cartridge useful in the process of the invention. The cartridge comprises a first inlet 101 for feeding the aqueous phase 101′ and a second inlet 102 for supplying the organic phase 102′. The aqueous phase and the organic phase are directed towards a mixing portion 103, for instance, a staggered herringbone micromixer 203 as illustrated in FIG. 2, wherein they are mixed (e.g. through laminar mixing in the case of the micromixer of FIG. 2 endowing to the formation of calibrated fluorocarbon cross-linkable vesicles.

The cross-linkable calibrated (per) fluorocarbon vesicles are then directed to the exit channel 104, from where they are collected in a suitable container (e.g. a vial).

Alternatively, said microfluidic cartridge can be equipped with an additional channel, for instance, placed between the mixing portion 103 and the exit channel 104, aimed at diluting, with a suitable solvent, the calibrated (per) fluorocarbon vesicles suspension before their direction to the exit channel 104 (i.e. in-line dilution).

A mixing portion 103 is generally characterized by suitable geometries/architecture able to enhance the microfluidic-mixing performance. In fact, the mixing process takes place into the peculiar micro-channel geometry of the mixing portion, which causes fluid streams to mix together on the way to exit the microfluidic cartridge.

Different types of mixing portions are available with different shapes or microstructures. Suitable examples of mixing portions can be classified as passive micromixers, such as T and Y shaped mixers (e.g. staggered herringbone micromixer or toroidal mixer), and the mixer using flow focusing; and active micromixers, such as mixer using pressure field disturbance, electrokinetic active micromixer and ultrasound active micromixer.

Preferred in the present invention is a staggered herringbone micromixer (FIG. 2), wherein the mixing of the two liquid phases is controlled by lamination-mixing or a toroidal micromixer.

During the mixing phase, the (per) fluorocarbon cross-linkable vesicles are formed and directed to the exit channel of the microfluidic cartridge or, alternatively, directed to an additional channel to dilute the vesicles before their direction to the exit channel.

The expression “exit (or outlet) channel” indicates the terminal portion of the microfluidic cartridge, toward which the just formed cross-linkable vesicles are directed from the mixing portion and from where it is possible to collect the formed suspension of cross-linked vesicles in a suitable container (e.g. a vial).

An example of microfluidic cartridge is the commercially available NxGen Cartridge, with or without in-line dilution, from Precision Nanosystems (Vancouver, Canada). These microfluidic cartridges can comprise either staggered herringbone or toroidal micromixers, both operating under non-turbulent conditions. For the manufacturing process, the microfluidic cartridge is mounted on a microfluidic instrument, generally equipped with a cartridge adapter, to host the microfluidic cartridge, and with containers (e.g. syringes or vials for continuous-flow injection) directly connected to the inlets of the microfluidic cartridge and specifically designed to pump the liquid phases into said inlets. Example of a microfluidic instrument is the NanoAssemblr® Benchtop Automated Instrument from Precision Nanosystems (Vancouver, Canada).

An aspect of the invention relates to a method for the preparation of an aqueous suspension of calibrated fluorocarbon cross-linked vesicles, said method comprising the steps of:
a) Preparing an aqueous phase wherein the temperature of said aqueous phase is at least of 10° C.;
b) Preparing an organic phase,
   wherein
   i) said aqueous phase comprises a cross-linkable amphiphilic peptide and said organic phase comprises a fluorocarbon
   or
   ii) said organic phase comprises a cross-linkable amphiphilic peptide and a fluorocarbon;
c) Injecting said aqueous phase in a first inlet and said organic phase in a second inlet of a microfluidic cartridge, thereby mixing said aqueous phase and said organic phase in a mixing portion of the microfluidic cartridge to obtain an aqueous suspension of calibrated fluorocarbon cross-linkable vesicles;
d) Collecting the aqueous suspension of calibrated fluorocarbon cross-linkable vesicles from an exit channel of the microfluidic cartridge, and
e) Cross-linking the cross-linkable amphiphilic peptides to obtain an aqueous suspension of calibrated fluorocarbon cross-linked vesicles.

According to a preferred embodiment, said aqueous phase comprises a cross-linkable amphiphilic peptide and said organic phase comprises a fluorocarbon, preferably said fluorocarbon being a perfluorocarbon.

According to the disclosed method, it is possible to obtain an aqueous suspension of calibrated (per)fluorocarbon cross-linkable vesicles by a single passage of the liquid phases through the two-channel microfluidic system.

In an embodiment said method for the preparation of an aqueous suspension of calibrated (per)fluorocarbon vesicles is the microfluidic technique, wherein said calibrated (per)fluorocarbon vesicles (Z-average diameter comprised between 100 and 1000 nm) have a polydispersity index (PDI) lower than 0.25, preferably lower than 0.20, more preferably lower than 0.15, even more preferably lower than 0.1.

### Aqueous Phase

The “aqueous phase” typically comprises an aqueous liquid component, including, for instance, water, aqueous buffered solutions or aqueous isotonic solutions.

Suitable examples of aqueous buffered solutions are phosphate-buffered saline (i.e. PBS buffer), sodium acetate buffer, tris(hydroxymethyl)aminomethane buffer (i.e. TRIS buffer) or a mixture thereof.

Suitable examples of isotonic solutions are Ringer solution, Ringer's lactate solution, saline, oral rehydration solution or a mixture thereof.

Preferably the aqueous liquid component has a pH lower than 7.0, more preferably lower than 6.5, still more preferably lower than 6.0, more preferably lower than 5.5., more preferably lower than 5, more preferably lower than 4.5, still more preferably lower than 4.0, up to e.g. 0, preferably 1.0.

Preferably the aqueous liquid component is sodium acetate buffer.

According to a preferred embodiment, said aqueous phase comprises a cross-linkable amphiphilic peptide.

For instance, a cross-linkable amphiphilic peptide can be admixed with an aqueous component through traditional techniques (e.g. stirring) in order to prepare the aqueous phase to be injected into the first inlet of the microfluidic cartridge.

At step a) the aqueous phase comprises a cross-linkable amphiphilic peptide preferably at a concentration ranging between 0.0003 mmol/mL and 0.006 mmol/mL, more preferably between 0.0006 mmol/mL and 0.004 mmol/mL, still more preferably between 0.0028 mmol/mL and 0.0034 mmol/mL.

In a further embodiment, the cross-linkable amphiphilic peptide comprised in the aqueous phase at step a) of the disclosed method is the compound of formula IV (SEQ ID NO: 4):

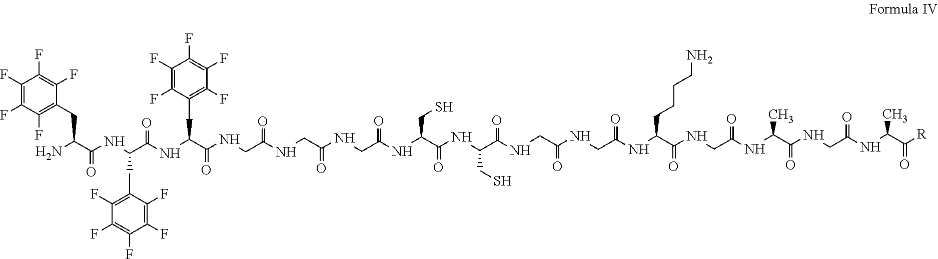

The Applicant has unexpectedly observed that the temperature at which said aqueous phase (in particular when comprising a cross-linkable amphiphilic peptide) is injected into the microfluidic cartridge, has remarkable effects on the sizes and polydispersity values of the microfluidically-obtained cross-linked vesicles. For example, when the aqueous phase is injected having a temperature lower than room temperature (e.g. 4° C.) negative results in terms of dimensions and polydispersity are achieved at the end of the process. On the contrary, when the aqueous phase is injected having a temperature close to room temperature (i.e. 20° C.) the final calibrated (per)fluorocarbon cross-linked vesicles are characterized by low size and low PDI values, giving a monodisperse composition. This monodispersity can positively influence the properties of the formulation, such as the stability.

In a preferred embodiment, at step a) the temperature of said aqueous phase is at least 15° C., more preferably at least of 18° C., still more preferably at least of 19° C. The temperature of the aqueous phase is typically not higher than the boiling point of the components of said organic phase, preferably not higher than the boiling point of the component of said organic phase having the lowest boiling point. For example the temperature of the aqueous phase is lower than 60° C., more preferably lower than 30° C., still more preferably lower than 25° C., preferably lower than 22° C., more preferably the temperature is at least 20° C. Preferably the temperature is at least 20° C.±1 (i.e. room temperature).

### Organic Phase

The “organic phase” typically comprises an organic solvent, preferably miscible with water, including, for instance, methanol, ethanol, isopropanol, acetonitrile, DMF, DMSO and acetone. Preferably the organic solvent is ethanol.

In the present invention the expression “organic solvent miscible with water” indicates an organic solvent capable of mixing in any ratio (e.g. any concentration) with water without separation of the two phases, i.e. forming a homogeneous solution.

For instance, C₁-C₃ alchools, such as methanol, ethanol and propanol, are very soluble in water due to the hydrogen bonding engaging the hydroxyl groups in the alcohol molecules and the water molecules. However as the length of the hydrocarbon chain increases, the solubility in water decreases leading to a low miscibility of the two liquids that, if mixed, will form two immiscible layers.

According to a preferred embodiment, said organic phase comprises a fluorocarbon or a mixture of different fluorocarbons dispersed in the organic solvent. Preferably the fluorocarbons are perfluorocarbons.

Suitable examples of (per)fluorocarbons are those mentioned above.

As an example, a fluorocarbon can be admixed with an organic solvent through traditional techniques (e.g. stirring) in order to prepare the organic phase to be injected into the second inlet of the microfluidic cartridge.

In a further embodiment, at step b) the organic phase comprises a fluorocarbon at a concentration ranging between 0.003 mmol/mL and 0.142 mmol/mL, more preferably between 0.011 mmol/mL and 0.085 mmol/mL, still more preferably between 0.013 mmol/ml to 0.057 mmol/mL.

In an alternative embodiment, said organic phase comprises a cross-linkable amphiphilic peptide and a fluorocarbon.

For instance, said organic phase comprises
i) A cross-linkable amphiphilic peptide at a concentration ranging between 0.0003 mmol/mL and 0.006 mmol/mL, more preferably between 0.0006 mmol/mL to 0.004 mmol/mL, still more preferably between 0.0028·10⁻³ mmol/mL and 0.0034 mmol/mL; and
ii) a (per)fluorocarbon at a concentration ranging between 0.003 mmol/mL to 0.142 mmol/mL, more preferably between 0.011 mmol/mL and 0.085 mmol/mL, still more preferably between 0.013 mmol/mL to 0.057 mmol/mL.

In an embodiment, the organic solvent is a polar organic solvent.

The expression “polar organic solvent” has its conventional meaning in the chemical field. Solvents can be classified by their relative polarity (rp): for example, water is the most polar solvent and it is characterized by a relative polarity of 1. On the contrary non-polar solvents have low value of relative polarity, such as dimethylformamide (DMF) with a relative polarity value of 0.386.

The Applicant observed that a low or medium-low polarity of the organic solvent may negatively affect the monodispersity of the final suspension of cross-linked vesicles. For instance, the use of organic solvents having a relatively low polarity, such as dimethylformamide (rp 0.386) or isopropyl alcohol (rp 0.546), may result in vesicles size distributions with relatively high values of PDI (e.g. higher than 0.25). On the contrary, the use of organic solvents having a relatively higher polarity, such as methanol (rp 0.762) or ethanol (rp 0.654), generally allow to obtain particle size distributions with lower PDI values, indicating a good monosdispersity of the samples.

Moreover, also the cross-linked vesicles sizes may be influenced by the polarity of the organic phase. In particular, sizes were inversely proportional to the polarity of the organic phase: high polar solvents, like methanol and ethanol, led to lower sizes than low and medium-low polar solvents, like DMF and isopropyl alcohol.

In a preferred embodiment, the organic solvent is a polar organic solvent, said solvent having a polarity comprised between 0.60 and 0.80, preferably comprised between 0.63 and 0.78, still more preferably comprised between 0.65 and 0.77.

In a further embodiment, said organic solvent is selected from methanol, ethanol and mixture thereof. Preferably the organic phase is ethanol.

At step b) the temperature of the organic phase is preferably lower than room temperature (e.g. about 4° C.) to avoid vaporization of fluorocarbons having a boiling point close to room temperature.

In an embodiment, at the step b) the temperature of the organic phase is typically lower than 25° C., preferably lower than 10° C., more preferably the temperature is about 5±2° C. Said temperature is preferably not lower than 2° C.

In a further preferred embodiment, at step a) said aqueous phase comprises a cross-linkable amphiphilic peptide and has a temperature of at least 10° C. and at step b) the temperature of the organic phase is 5±2° C.

### Total Flow Rate (TFR) and Flow Rate Ratio (FRR)

The method of the present invention allows controlling the (per)fluorocarbon cross-linked vesicles characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 200 mL/min, preferably 2 and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 1:

$$Flow rate ratio = \frac{volume of aqueous phase}{volume of organic phase} \qquad Eq . 1$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1, more preferably the FRR is 1:1.

In the present invention, the respective concentrations of both cross-linkable amphiphilic peptide and (per)fluorocarbon and the FRR can be purposely tuned in order to obtain a molar ratio between said cross-linkable amphiphilic peptide and said (per)fluorocarbon suitable to assure the stability of the cross-linked vesicle (i.e certain values of sizes and PDI). The molar ratio between said cross-linkable amphiphilic peptide and said (per)fluorocarbon is preferably comprised between 0.002 and 7.000. More preferably the ratio is not higher than 6.100, not higher than 5.000, not higher than 4.000, not higher than 3.000, not higher than 2.000, even more preferably not higher than 1.500. More preferably the molar ratio is not lower than 0.001, not lower than 0.004, even more preferably not lower than 0.010.

### Step e) Cross-Linking Step

The step e) of the method of the present invention comprises cross-linking the cross-linkable amphiphilic peptides to obtain an aqueous suspension of calibrated fluorocarbon cross-linked vesicles.

In the present description and claims, the expression “cross-linking” indicates the process of forming covalent bonds between cross-linkable moieties comprised in adjacent cross-linkable amphiphilic peptides in order to bind cross-linkable amphiphilic peptides molecules together. Preferably said cross-linkable moieties are cross-linkable amino acids comprised in adjacent cross-linkable amphiphilic peptides.

In a preferred embodiment, said cross-linkable amino acids are cysteine amino acids comprised in adjacent cross-linkable amphiphilic peptides. Said cysteine amino acids are cross-linked via disulfide cross-linking groups (—S—S—).

In a preferred embodiment, step e) comprises contacting said aqueous suspension of calibrated fluorocarbon cross-linkable vesicles with an oxidizing source able to induce the cross-linking of the cross-linkable amphiphilic peptides to obtain an aqueous suspension of calibrated fluorocarbon cross-linked vesicles.

The oxidizing source is contacted with the freshly prepared aqueous suspension of calibrated (per)fluorocarbon cross-linkable vesicles for a time sufficient to induce the cross-linking of the cross-linkable amphiphilic peptides comprised in the outer layer of the vesicles.

In an embodiment, said suitable time is typically of at least one minute, preferably at least 30 minutes, more preferably at least one hour. The time typically does not exceed 24 hours, preferably less than 18 hours, more preferably less than 15 hours.

According to another embodiment, the oxidizing source is contacted with the freshly prepared aqueous suspension of cross-linkable vesicles for a time sufficient to induce a degree of cross-linking of the cross-linkable amphiphilic peptide molecules of higher than 80%, preferably at least 85%, more preferably at least 90%. Preferably the degree of cross-linking is 100%.

Suitable examples of oxidizing sources are oxidizing solutions, oxidizing gas or a mixture thereof.

The term “oxidizing solution” indicates any aqueous solution which may cause or contribute to induce the formation of covalent bonds between cross-linkable amino acids, e.g. between the thiol groups of cysteine amino acids comprised in adjacent cross-linkable amphiphilic peptides.

Suitable examples of oxidizing solutions are water, aqueous solution of DMSO, basic solutions comprising halogens, aqueous hydrogen peroxide comprising iodine and selenide-based catalyst in presence of air.

In an embodiment, the oxidizing solution is preferably an aqueous solution of DMSO, wherein the concentration of said DMSO is typically of at least 0.1%, preferably at least 1%, more preferably at least 1.5%. The concentration of said DMSO typically does not exceed 10%, preferably less than 5%, more preferably less than 3.5%.

In a preferred embodiment, the oxidizing solution is preferably an aqueous solution of DMSO at 2.5%.

The term “oxidizing gas” indicates any gas which may induce or contribute to the formation of covalent bonds between cross-linkable amino acids, e.g. between the thiol groups of cysteine amino acids comprised in adjacent cross-linkable amphiphilic peptides.

Suitable examples of oxidizing gas are oxygen, air or suitable mixtures of gas comprising oxygen.

In a preferred embodiment, the oxidizing gas is air.

The oxidizing gas is contacted with the freshly prepared aqueous suspension of calibrated (per)fluorocarbon cross-linkable vesicles for a time of at least one minute, preferably at least 30 minutes, more preferably at least one hour. The time typically does not exceed 24 hours, preferably less than 10 hours, more preferably less than 3 hours.

For instance, at the end of the microfluidic process, an oxidizing gas, e.g. air, can be injected by bubbling into the oxidizing solution comprising the dialysis device including the aqueous suspension of calibrated PFC cross-linkable vesicles.

In a still more preferred embodiment, said oxidizing source comprises a mixture of an oxidizing solution and an oxidizing gas.

Preferably, said oxidizing source comprises a mixture of an aqueous oxidizing solution and air.

In a preferred embodiment, the step e) is a dialysis procedure comprising contacting an aqueous suspension of calibrated fluorocarbon cross-linkable vesicles with an oxidizing source able to induce the cross-linking of the cross-linkable amphiphilic peptides to obtain an aqueous suspension of calibrated fluorocarbon cross-linked vesicles

Preferably said oxidizing source is an oxidizing solution as described above.

Alternatively, said oxidizing source comprises a mixture of an oxidizing solution and an oxidizing gas as described above.

In the present description, the expression “dialysis” indicates a procedure for promoting the cross-linking of the freshly prepared aqueous suspension of calibrated (per)fluorocarbon cross-linkable vesicles, through the mechanisms described above.

According to this embodiment, after the collection from the microfluidic cartridge, the aqueous suspension of calibrated (per)fluorocarbon cross-linkable vesicles is loaded inside a dialysis device comprising a semi-permeable membrane which is contacted with an oxidizing source, for instance by suspending said dialysis device in a large volume of an oxidizing solution, as defined above, into which is simultaneously injected an oxidizing gas.

Suitable examples of dialysis devices comprising a semi-permeable membrane can be traditional dialysis tubings or advanced dialysis devices, such as dialysis cassettes, dialysis flasks or dialysis plates. The permeability of said semi-permeable membrane is such that the oxidizing solution can contact the calibrated (per)fluorocarbon cross-linkable vesicles, loaded inside the dialysis device, consequently inducing their cross-linking. Said dialysis procedure allows efficient recovery of an aqueous suspension of calibrated (per)fluorocarbon cross-linked vesicles.

In a preferred embodiment, the step e) is a dialysis procedure comprising the steps of:
eⁱ) loading the aqueous suspension of calibrated (per)fluorocarbon cross-linkable vesicles inside a dialysis device comprising a semi-permeable membrane;
eⁱⁱ) contacting said loaded dialysis device with an oxidizing solution and
eⁱⁱⁱ) collecting an aqueous suspension of calibrated (per)fluorocarbon cross-linked vesicles from the device.

In an embodiment, at step eⁱⁱ) the oxidizing solution is an aqueous solution of DMSO at 2.5%.

In another embodiment, step eⁱⁱ) comprises contacting said dialysis device with a mixture of an oxidizing solution and an oxidizing gas. Preferably, said mixture comprises an aqueous solution of DMSO at 2.5% and air.

Preferably step eⁱⁱ) comprises contacting said dialysis device with an aqueous solution of DMSO at 2.5% for 12 hours and air for the first hour.

For instance, the aqueous suspension of calibrated (per)fluorocarbon cross-linkable vesicles may be loaded inside a dialysis device comprising a semi-permeable membrane and then said device can be contacted with an aqueous solution of DMSO at 2.5% for 12 hours, under constant air bubbling for the first hour.

In a still further embodiment, step eⁱⁱ) is repeated from 1 up to 5 times, preferably up to 3 times, more preferably step eⁱⁱ) is repeated 2 times.

At the end of each step eⁱⁱ), the oxidizing solution can be replaced with fresh oxidizing solution or alternatively with a different oxidizing solution. In an embodiment each step eⁱⁱ) can be performed by contacting the dialysis device with the same oxidizing solution, e.g. aqueous solution of DMSO at 2.5%. Said oxidizing solution may be replaced with a freshly prepared solution at the beginning of each repeated step.

Alternatively, each step eⁱⁱ) can be performed by contacting the dialysis device with a different oxidizing solution. For instance, the aqueous suspension of calibrated (per)fluorocarbon cross-linkable vesicles may be loaded inside a dialysis device comprising a semi-permeable membrane and then said device can be contacted with an aqueous solution of DMSO at 2.5% for 12 hours, under constant air bubbling for the first hour and sequentially it can be contacted with water for 2 hours.

In some embodiments, at step eⁱⁱ) DMSO is replaced with water to reduce the amount of DMSO in the final aqueous suspension of calibrated cross-linked vesicles.

As observed by the Applicant, following the microfluidic preparation of the cross-linkable PFC vesicles and their cross-linking as above described, the degree of cross-linking is unexpectedly high, typically the degree of cross-linking is higher than 80%, preferably at least 85%, more preferably at least 90%, still more preferably at least 95%. Preferably the degree of cross-linking is 100%.

Optional Step d′) Dilution

In an embodiment, the method of preparation further comprises an optional step d′), which comprises diluting the aqueous suspension of calibrated (per)fluorocarbon cross-linkable vesicles.

In an embodiment, the optional step d′) is carried out sequentially to step d). For example, the optional step d′) can be performed between step d) and step e), in particular after the step d), for instance within 5 minutes from collecting the sample from the microfluidic cartridge, and before starting the cross-linking phase.

The Applicants have unexpectedly observed that a dilution step carried out after the production of the calibrated cross-linkable vesicles using a microfluidic cartridge, has a favorable effect on the initial size and initial monodispersity. Indeed, without dilution, the cross-linkable vesicles size was larger than with a dilution.

As indicated above, the expressions “initial monodispersed distribution” and “initial sizes” refer to the values of monodispersity and sizes of the calibrated (per)fluorocarbon cross-linkable vesicles composition collected from the exit channel of the microfluidic cartridge at the step d) of the disclosed method of preparation.

In the present description and claims the term “dilution” refers to the process of reducing the concentration of calibrated (per)fluorocarbon cross-linkable vesicles in the suspension, by adding a suitable amount of aqueous component.

A suitable amount of aqueous component corresponds to the quantity of aqueous solution necessary to reduce the concentration of the calibrated vesicles in the aqueous suspension from 2 to 10-folds.

In a preferred embodiment, the optional step d′) of the present method comprises diluting the aqueous suspension of calibrated fluorocarbon vesicles from 1 to 20-folds, preferably from 2 to 10-folds, still more preferably from 3-to 8-folds e.g. about 5-fold.

Suitable aqueous components are water or oxidizing solutions as described above.

Preferably said aqueous components are oxidizing solutions, preferred being an aqueous solution of DMSO.

In a preferred embodiment, the step d′) of the present method comprises diluting the suspension of calibrated (per)fluorocarbon cross-linkable vesicles with an aqueous solution of DMSO.

In a preferred embodiment, the concentration of DMSO in the aqueous solution is typically of at least 0.1%, preferably at least 1%, more preferably at least 1.5%. The concentration of said DMSO typically does not exceed 10%, preferably less than 5%, more preferably less than 3.5%.

In an embodiment, the diluting step can be performed directly inside the microfluidic cartridge (i.e. in-line dilution), by way of an additional channel (e.g. placed between the mixing portion 103 and the exit channel 104 in FIG. 1) suitable for diluting the calibrated fluorocarbon cross-linkable vesicles suspension with the desired aqueous component before their direction to the exit channel and before the cross-linking step e).

### Optional Step f: Washing

In a further embodiment, the method of the invention may comprise optional step f), which comprises washing the obtained aqueous suspension of calibrated fluorocarbon cross-linked vesicles.

According to this embodiment, after the cross-linking phase, the calibrated (per)fluorocarbon cross-linked vesicles are treated using suitable washing techniques, in order to remove fluorocarbon-free assembly.

In the present description, the term “washing” indicates any operation carried out on the freshly prepared (per)fluorocarbon cross-linked vesicles suspension, finalized to remove (or substantially reduce the amount of) fluorocarbon-free assemblies.

In the present description, the expression “fluorocarbon free-assembly” indicates an assembly comprising cross-linkable amphiphilic peptides spontaneously assembled in a particle due to hydrophobic interactions. Said assemblies may be formed during the microfluidic process and may be present in the aqueous suspension of calibrated fluorocarbon cross-linked vesicles at the end of the preparation process, but due to their lower sizes, they contribute to forming a second population of particles, endowing to a higher value of PDI. For example, the fluorocarbon-free assemblies are characterized by sizes comprised between 50 nm and 150 nm.

The Applicant observed that performing a washing procedure on the freshly prepared calibrated (per)fluorocarbon cross-linked vesicles suspension led to lower values of PDI, increasing the stability of the microfluidically-obtained composition.

Suitable washing techniques comprise, for instance, centrifugation, ultracentrifugation, filtration and decantation.

The optional step f) is carried out sequentially to step e), for example, it is carried out after the completion of the cross-linking step, e.g. within 5 minutes.

In a preferred embodiment, the washing step f) comprises the steps of:
fⁱ) centrifuging the aqueous suspension of calibrated fluorocarbon cross-linked vesicles obtained from step e);
fⁱⁱ) separating the supernatant phase comprising fluorocarbon-free assemblies, and
fⁱⁱⁱ) adding an aqueous solution to obtain an aqueous suspension of calibrated fluorocarbon cross-linked vesicles.

The washing in step f) can be carried out from 1 time to 10 times, preferably is performed from 2 times to 5 times, still more preferably is performed 3 times.

The duration of the centrifugation at step fⁱ is of at least 1 minute, preferably at least 3 minutes, more preferably at least 4 minutes. The duration of the centrifugation is typically lower than 10 minutes, preferably lower than 7 minutes, more preferably the duration is 5minutes.

The temperature at which is performed the centrifugation at step fⁱ is typically of at least 1° C., preferably at least 2° C., more preferably at least 3° C. Said temperature does not exceed 25° C., preferably is lower than 20° C., more preferably is lower than 10° C.

The rotation at which is performed the centrifugation at step f is comprised between 1000 g and 10000 g, preferably between 2000 g and 6000 g, still more preferably the rotation is 5000 g.

The aqueous solution of step fⁱ is preferably physiologically acceptable, comprising water (preferably sterile water), aqueous solutions such as saline (which may advantageously be balanced so that the final product for injection is not hypotonic), or solutions of one or more pharmaceutical excipients.

Suitable examples of pharmaceutical excipients are tonicity adjusting substances. Tonicity adjusting substances comprise salts or sugars, sugar alcohols, glycols or other non-ionic polyol materials (e.g. glucose, sucrose, trehalose, sorbitol, mannitol, glycerol, polyethylene glycols, propylene glycols and the like), chitosan derivatives, such as carboxymethyl chitosan, trimethyl chitosan or jellifying compounds, such as carboxymethylcellulose, hydroxyethyl starch, hydrolyzed collagen, or dextran.

Preferred is a glucose solution. The glucose aqueous solution typically comprises glucose at a concentration of at least 1%, preferably at least 2.5%, more preferably at least 3%. The glucose concentration typically does not exceed 20%, preferably is less than 10%, more preferably is less than 7%.

### Aqueous Suspension of a Calibrated (per)fluorocarbon Cross-Linked Vesicles

An aspect of the invention relates to an aqueous suspension comprising a plurality of calibrated (per)fluorocarbon cross-linked vesicles, obtained according to the process as above defined.

An aspect of the invention relates to an aqueous suspension comprising a plurality of calibrated fluorocarbon cross-linked vesicles obtainable by the process comprising the steps of:
a) Preparing an aqueous phase wherein the temperature of said aqueous phase is at least of 10° C.;
b) Preparing an organic phase, wherein
   i) said aqueous phase comprises a cross-linkable amphiphilic peptide and said organic phase comprises a fluorocarbon or
   ii) said organic phase comprises a cross-linkable amphiphilic peptide and a fluorocarbon;
c) Injecting said aqueous phase in a first inlet and said organic phase in a second inlet of a microfluidic cartridge, thereby mixing said aqueous phase and said organic phase in a mixing portion of the microfluidic cartridge to obtain an aqueous suspension of calibrated fluorocarbon cross-linkable vesicles;
d) Collecting the aqueous suspension of calibrated fluorocarbon intermediate cross-linked vesicles from the exit channel of the microfluidic cartridge, and
e) Cross-linking the cross-linkable amphiphilic peptides to obtain an aqueous suspension of calibrated fluorocarbon cross-linked vesicles,

wherein said cross-linked vesicles have a z-average diameter comprised between 100 nm and 1000 nm and a polydispersity lower than 0.25.

In a preferred embodiment said cross-linked vesicles have a degree of cross-linking higher than 80%, preferably at least 85%, more preferably at least 90%, still more preferably at least 95%. Preferably the degree of cross-linking is 100%.

In the present description and claims, the term “plurality of calibrated PFC cross-linked vesicles” refers to a population of cross-linked vesicles characterized by a calibrated distribution, meaning that substantially all the vesicles have substantially similar sizes.

The expression “calibrated distribution” indicates a polydispersity (PDI) of a certain population of cross-linked vesicles (e.g. with a z-average diameter comprised between 100 and 1000 nm) with a polydispersity index (PDI) lower than 0.25, preferably lower than 0.2, more preferably lower than 0.15, even more preferably lower than 0.1.

The term “polydispersity” (PDI) refers to a dimensionless measure of the broadness of the size distribution calculated from the cumulants analysis, wherein said cumulants analysis, defined in the International Standard on Dynamic Light Scattering ISO13321 (1996) and ISO22412 (2008), gives a mean particle size (z-average) and an estimate of the width of the distribution (polydispersity index).

For instance, a polydispersity higher than 0.7 indicates a very broad distribution of particles sizes, while a value lower than 0.08 indicates a nearly monodisperse sample characterized by a monomodal distribution. The polydispersity can be measured with the dynamic light scattering technique (DLS), by using for instance the Malvern Zetasizer Nano-ZS instrument (Malvern Instruments Ltd., UK).

The “z-Average Diameter (ZD)” is defined as the intensity-weighted mean diameter derived from the cumulants analysis. In other words, it relates to the average of calibrated vesicles size dispersed in the aqueous suspension measured through the dynamic light scattering technique (DLS).

In the present invention, the z-average diameter is comprised between 100 nm and 1000 nm, preferably between 120 and 600, more preferably between 150 and 400.

The aqueous suspension comprising a plurality of calibrated (per)fluorocarbon cross-linked vesicles further comprises an aqueous liquid component as above defined. Suitable aqueous components for said aqueous suspension are preferably physiologically acceptable comprising water (preferably sterile water), saline (which may advantageously be balanced so that the final product for injection is not hypotonic), aqueous buffered solution or aqueous isotonic solution.

A further embodiment relates to said aqueous suspension comprising a plurality of calibrated fluorocarbon cross-linked vesicles in admixture with a pharmaceutically acceptable excipient, preferably obtained by the above described microfluidic method.

Examples of pharmaceutically acceptable excipients are tonicity adjusting substances. Tonicity adjusting substances comprise salts or sugars, sugar alcohols, glycols or other non-ionic polyol materials (e.g. glucose, sucrose, trehalose, sorbitol, mannitol, glycerol, polyethylene glycols, propylene glycols and the like), chitosan derivatives, such as carboxymethyl chitosan, trimethyl chitosan or jellifying compounds, such as carboxymethylcellulose, hydroxyethyl starch, hydrolyzed collagen or dextran.

In a preferred embodiment said pharmaceutically acceptable excipient is glucose.

In another embodiment, the glucose aqueous solution typically comprises glucose at a concentration of at least 1%, preferably at least 2.5%, more preferably at least 3%. The glucose concentration typically does not exceed 20%, preferably is less than 10%, more preferably is less than 7%.

For instance, the aqueous suspension comprising a plurality of calibrated (per)fluorocarbon cross-linked vesicles can be admixed with said pharmaceutically acceptable excipient during the step e) of cross-linking (e.g. during dialysis).

In an alternative embodiment, the step e) is a dialysis procedure comprising the steps of:
eⁱ) loading the aqueous suspension of calibrated (per)fluorocarbon cross-linkable vesicles inside a dialysis device comprising a semi-permeable membrane;
eⁱⁱ) contacting said loaded dialysis device with an aqueous solution of DMSO at 2.5% for 12 hours under constant air bubbling for the first hour;
e^{iV}) contacting said loaded dialysis device with water for 2 hours;
e^{v}) contacting said loaded dialysis device with an aqueous solution comprising a pharmaceutically acceptable excipient for 4 hours and
eⁱⁱⁱ) collecting an aqueous suspension comprising a plurality of calibrated (per)fluorocarbon cross-linked vesicles admixed with an acceptable pharmaceutical excipient from the dialysis device.

Alternatively, the aqueous suspension comprising a plurality of calibrated (per)fluorocarbon cross-linked vesicles can be admixed with said pharmaceutically acceptable excipient during the optional washing step f).

In an optional embodiment, step f) comprises the steps of:
fⁱ) centrifuging the aqueous suspension of calibrated (per)fluorocarbon cross-linked vesicles obtained from step e);
fⁱⁱ) separating the supernatant phase comprising fluorocarbon-free assemblies and residue compounds, and
fⁱⁱⁱ) adding an aqueous solution comprising a pharmaceutically acceptable excipient to obtain an aqueous suspension of calibrated (per)fluorocarbon cross-linked vesicles admixed with said pharmaceutically acceptable excipient.

Preferably said pharmaceutically acceptable excipient is glucose.

### Optional Step g): Freezing

A still further embodiment of the invention relates to a method for the preparation of an aqueous suspension of calibrated fluorocarbon cross-linked vesicles, comprising an optional step g), subsequent to step e), comprising freezing the suspension of calibrated (per)fluorocarbon cross-linked vesicles.

In an embodiment, said step g) comprises freezing the suspension of calibrated (per)fluorocarbon vesicles at a temperature comprised between −60° C. and 0° C., preferably comprised between −40° C. and −10° C., still more preferably the temperature is −30° C.

The frozen suspension can then be stored at a temperature comprised between −30° and −10° C., preferably −20° C.

### Use

The acoustic droplet vaporization (ADV) is a phenomenon through which perfluorocarbon cross-linked vesicles can be converted into gas microbubbles upon exposure to ultrasound energy beyond the vaporization threshold.

When administered in-vivo, perfluorocarbon cross-linked vesicles present many advantages with respect to traditional microbubbles, such as inertness, relatively low toxicity, relative stability in circulation, immiscibility in water, and low surface tension (Sheeran et al, 2011). Once vaporized, the generated microbubbles can be effectively used in either imaging or therapeutic applications with ultrasound, including sonopermeabilization, thermal ablation, blood brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging since these agents can be activated and deactivated on demand by applying intermittent acoustic pulses.

A further aspect relates to an aqueous suspension comprising a plurality of calibrated fluorocarbon cross-linked vesicles obtained according to the process as above defined, wherein said cross-linked vesicles have a z-average diameter comprised between 100 nm and 1000 nm and a polydispersity lower than 0.25, for use in a diagnostic and/or therapeutic treatment.

Preferably said cross-linked vesicles have a degree of cross-linking higher than 80%, preferably at least 85%, more preferably at least 90%, still more preferably at least 95%. Preferably the degree of cross-linking is 100%.

Diagnostic treatment includes any method where the use of the cross-linked vesicles allows enhancing the visualisation of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation), US aberration correction and super-resolution imaging.

Therapeutic treatment includes any method of treatment of a patient. In preferred embodiments, the treatment comprises the combined use of ultrasounds and (per)fluorocarbons vesicles either as such (e.g. in ultrasound-mediated thrombolysis, high intensity focused blood-brain ultrasound ablation, barrier permeabilization, immunomodulation, neuromodulation, radiosensitization) or in combination with a therapeutic agent (i.e. ultrasound-mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the vesicles may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound-mediated delivery).

The following examples will help to further illustrate the invention.

### EXAMPLES

### Example 1

General Procedure for the Preparation of PFC Cross-Linked Vesicles using a Microfluidic Platform

Perfluorocarbon cross-linked vesicles were formulated with a NanoAssemblr™ Benchtop automated instrument from Precision Nanosystems (Vancouver, Canada) equipped with a staggered herringbone micromixer (SHM) allowing size-controlled self-assemblies. Briefly, an aqueous phase comprising a cross-linkable amphiphilic peptides (CAP) was injected into the first inlet whereas the organic phase composed of PFC dissolved in ethanol was injected into the second inlet of the microfluidic cartridge (FIG. 1). The aqueous phase was stored at room temperature (i.e. 20° C.) and the organic phase was placed into an ice bath at about 4° C. before the cross-linkable vesicles formulation. Microscopic characteristics of the channels are engineered to cause an accelerated mixing of the two fluid streams in a controlled fashion. The microfluidic process settings namely the Total Flow Rate (TFR, in mL/min), and the Flow Rate Ratio (FRR), were varied to control the cross-linkable vesicles characteristics.

The aqueous suspension of calibrated cross-linkable vesicles was collected from the exit channel in a Falcon vial (15 mL) and then transferred to a Slide-A-Lyzer™ Dialysis Cassettes from Thermo Fisher Scientific and dialyzed against 2.5% aqueous DMSO overnight with air bubbles for the first hour. Subsequently, the suspension was further dialyzed against Milli-Q® water for 2 hours and against aqueous glucose 5% solution for 4 hours.

At the end of the procedure, an aqueous suspension of calibrated PFC cross-linked vesicles was obtained.

The specific type and amounts of materials are indicated in the preparations illustrated in the following examples.

### Example 2

### Effect of Using Aqueous Phases at Different Temperatures

Aqueous suspensions of calibrated perfluorocarbon cross-linked vesicles were prepared through microfluidic method as described in Example 1. The cross-linkable amphiphilic peptide of formula IV (CAP-AGA; SEQ ID N: 3) was used at a concentration of 1 mg/ml (i.e. 0.62 mM) in the aqueous phase, and perfluoropentane (PFP) at 10 μL/mL was used in the organic phase. The molar ratio between the cAP-AGA and PFP was 0.011. The TFR was set at 10 mL/min and the FRR at 1-1. In order to evaluate the effect of the temperature of the aqueous phase on the sizes and PDI values of the cross-linked vesicles, two different samples were prepared:
Sample 2A: at the injection, the temperature of the aqueous phase was 4° C.;
Sample 2B: at the injection, the temperature of the aqueous phase was 20° C. (i.e.room temperature).
   Then both samples 2A and 2B were treated as described in Example 1 in order to obtain the aqueous suspensions of calibrated fluorocarbon cross-linked vesicles.

Results

TABLE 1
Effect of the temperature of the aqueous
phase on the sizes and PDI values

| Sample | T° of the aqueous phase | Mean diameter [nm] | PDI |
|---|---|---|---|
| 2A | 4° C. | 1397 | 0.23 |
| 2B | 20° C. | 321 | 0.12 |

Results showed that the temperature at which the aqueous phase is injected in the microfluidic cartridge has a remarkable influence on the characteristics of the cross-linked vesicles. In particular, it emerged that injecting the aqueous phase with a temperature of 4° C. (i.e. ice cold), comprising the cross-linkable amphiphilic peptide, negatively affects the sizes and the monodispersity of the cross-linked vesicles suspension. On the contrary, injecting the aqueous phase having a temperature of about 20° C. (i.e room temperature) led to good results in terms of size and PDI.

### Example 3

### Effect of the Aqueous Phase Composition

Aqueous suspensions of calibrated fluorocarbon cross-linked vesicles were prepared through the microfluidic method as described in Example 1. The cross-linkable amphiphilic peptide of formula IV (CAP-AGA; SEQ ID N: 3) was used at a concentration of 1 mg/ml (i.e. 0.62 mM) in the aqueous phase, and perfluoropentane (PFP) at 10 μL/mL was used in the organic phase. The molar ratio between the cAP-AGA and PFP was 0.011. The TFR was set at 10 mL/min and the FRR was set at 1-1. In order to evaluate the effect of the aqueous phase composition on the size and PDI values of the cross-linked vesicles, four different samples were prepared:
Sample 3C: having water as aqueous phase and ethanol as organic phase (corresponding to Sample 2B);
Sample 3D: having sodium acetate buffer as aqueous phase and ethanol as organic phase;
Sample 3E: having TRIS buffer as aqueous phase and ethanol as organic phase;
Sample 3F: having PBS buffer as aqueous phase ethanol as organic phase.

Then all the samples (3C, 3D, 3E and 3F) were treated as described in Example 1 in order to obtain the aqueous suspensions of calibrated PFC cross-linked vesicles.

### Results

TABLE 2
Effect of the aqueous phase on the sizes and PDI values

| Sample | Aqueous phase | PDI |
|---|---|---|
| 3C | Water | 0.12 |
| 3D | Sodium acetate buffer (pH = 4) | 0.09 |
| 3E | Tris buffer (pH = 6) | 0.09 |
| 3F | PBS buffer (pH = 7.5) | 0.28 |

From the results, it emerged that using PBS buffer as aqueous phase led to negative influence on monodispersity of the cross-linked vesicles suspension. On the contrary, water, sodium acetate buffer and TRIS buffer were able to assure good results in terms of PDI.

Moreover, substantially higher sizes were obtained using PBS buffer as aqueous phase (data not shown).

### Example 4

### Effect of the Organic Phase Composition

Aqueous suspensions of calibrated fluorocarbon cross-linked vesicles were prepared through microfluidic method as described in Example 1. The cross-linkable amphiphilic peptide of formula IV (CAP-AGA; SEQ ID N: 3) was used at a concentration of 1 mg/ml (i.e. 0.62 mM) in the aqueous phase, and perfluoropentane (PFP) at 10 μL/mL was used in the organic phase. The molar ratio between the cAP-AGA and PFP was 0.011. The TFR was set at 10 mL/min and the FRR was set at 1-1. In order to evaluate the effect of the organic phase composition on the size and PDI values of the cross-linked vesicles, four different samples were prepared:
Sample 4G: having sodium acetate buffer (pH 4) as aqueous phase and dimethylformamide (DMF) as organic phase;
Sample 4H: having sodium acetate buffer (pH 4) as aqueous phase and methanol (MeOH) as organic phase;
Sample 41: having sodium acetate buffer (pH 4) as aqueous phase and ethanol (EtOH) as organic phase (corresponding to Sample 3D);
Sample 4L: having sodium acetate buffer (pH 4) as aqueous phase and isopropyl alcohol (iPrOH) as organic phase.

### Results

TABLE 3
Effect of the organic phase on the sizes and PDI values

| Sample | Organic phase | PDI |
|---|---|---|
| 4G | Dimethylformamide (DMF) | 0.36 |
| 4H | Methanol (MeOH) | 0.12 |
| 4I | Ethanol (EtOH) | 0.09 |
| 4L | Isopropyl alcohol (iPrOH) | 0.28 |

From the results, it emerged that using DMF and isopropyl alcohol (i.e. organic solvents with relatively low polarity) as organic solvents in the organic phase negatively affected the distribution of the cross-linked vesicles suspension. On the contrary ethanol and methanol (relatively high polarity) were able to assure good results in terms of PDI.

### Example 5

### Effect of the Dilution Step

Aqueous suspensions of calibrated fluorocarbon cross-linked vesicles were prepared through microfluidic method as described in Example 1. The cross-linkable amphiphilic peptide of formula IV (CAP-AGA; SEQ ID N: 3) was used at a concentration of 5 mg/ml in the aqueous phase and perfluoropentane (PFP) at 10 μL/mL was used in the organic phase. The molar ratio between the cAP-AGA and PFP was 0.055. The TFR was set at 10 mL/min and the FRR was set at 1-1. In order to evaluate the effect of the dilution step on the size and PDI values of the cross-linked vesicles, at the end of the microfluidic process, the obtained suspension was divided into two aliquots: the first aliquot (Sample 5A) was maintained as such and the second one (Sample 5B) was diluted 5 times in 2.5% aqueous DMSO.

Both samples 5A and 5B were then transferred to a Slide-A-Lyzer™ Dialysis Cassettes from Thermo Fisher Scientific and dialyzed against 2.5% aqueous DMSO overnight with air bubbles for the first hour. Subsequently, sample 5A and sample 5B were further dialyzed against Milli-Q® water for 2 hours and against aqueous glucose 5% solution for 4 hours.

At the end of the dialysis steps, the size and PDI of the cross-linked vesicles suspensions were directly measured by DLS.

### Results

TABLE 4
Effect of the dilution on the size and PDI values

| Sample | Dilution | Mean diameter [nm] | PDI |
|---|---|---|---|
| 5A | No | 588 | 0.19 |
| 5B | Yes | 222 | 0.08 |

As displayed in Table 4, results showed that the dilution of the PFC cross-linked vesicles suspension obtained through microfluidic process (Sample 5B) was able to significantly reduce the size and to further improve the PDI in comparison to the not diluted sample (Sample 5A).

### Example 6

### Effect of the Solvent in the Dilution Step

Aqueous suspensions of calibrated fluorocarbon cross-linked vesicles were prepared through microfluidic method as described in Example 1. The cross-linkable amphiphilic peptide of formula IV (CAP-AGA; SEQ ID N: 3) was used at a concentration of 1 mg/ml in the aqueous phase, and perfluoropentane (PFP) at 10 μL/mL was used in the organic phase. The molar ratio between the APS-AGA and PFP was 0.011. The TFR was set at 10 mL/min and the FRR was set at 1-1. In order to evaluate the effect of the solvent used in the dilution step on the size and PDI values of the cross-linked vesicles, at the end of the microfluidic process, the obtained suspension was divided into two aliquots: the first aliquot (Sample 6C) was diluted 5 times in water and the second one (Sample 6D, corresponding to Sample 2B and 3C) was diluted 5 times in 2.5% aqueous DMSO.

Both samples 6C and 6D were then transferred to a Slide-A-Lyzer™ Dialysis Cassettes from Thermo Fisher Scientific and dialyzed against 2.5% aqueous DMSO overnight with air bubbles for the first hour. Subsequently, samples 6A and 6B were further dialyzed against Milli-Q® water for 2 hours and against aqueous glucose 5% solution for 4 hours.

At the end of the dialysis steps, the size and PDI of the cross-linked vesicles suspensions were directly measured by DLS.

### Results

TABLE 5
Effect of the solvent used in the dilution
step on the size and PDI values

| Sample | Solvent for the dilution | Mean diameter [nm] | PDI |
|---|---|---|---|
| 6C | Water | 409 | 0.26 |
| 6D | 2.5% DMSO | 321 | 0.12 |

Results showed that diluting the aqueous suspension of cross-linkable vesicles obtained through microfluidic process with water (Sample 6C) results in a formulation with a relatively high PDI. On the contrary, performing the dilution with an aqueous solution of DMSO at 2.5% (Sample 6D) led to good size and PDI values.

### Example 7

Effect of the Molar Ratio Nr=N_{peptide}/N_{PFC}

Aqueous suspensions of calibrated fluorocarbon cross-linked vesicles were prepared through microfluidic method as described in Example 1. In particular, in order to evaluate the effect of the molar ratio Nr between the cross-linkable amphiphilic peptide and the PFC on the size and PDI values of the cross-linked vesicles, five different samples were prepared:
Sample 7A having a Nr=0.011, obtained co-injecting into the microfluidic cartridge-an aqueous solution of cAP-AGA at 1 mg/ml (in Milli-Q water) and an organic solution of PFP (10 μL/mL). The TFR was set at 10 mL/min and the FRR was set at 1-1.
Sample 7B having a Nr=0.055, obtained co-injecting into the microfluidic cartridge an aqueous solution of cAP-AGA at 5 mg/ml (in Milli-Q water) and an organic solution of PFP (10 μL/mL). The TFR was set at 10 mL/min and the FRR was set at 1-1.
Sample 8A having a Nr=0.110 obtained co-injecting into the microfluidic cartridge an aqueous solution of cAP-AGA (SEQ ID N: 3) at 5 mg/ml (in Milli-Q water) and an organic solution of PFP at 5 μL/mL. The TFR was set at 10 mL/min and the FRR was set at 1-1.
Sample 8B having a Nr=0.220, obtained co-injecting into the microfluidic cartridge an aqueous solution of cAP-AGA at 5 mg/ml (in Milli-Q water) and an organic solution of PFP (5 μL/mL). The TFR was set at 10 ml/min and the FRR was set at 2-1.
Sample 8C having a Nr=0.330, obtained co-injecting into the microfluidic cartridge an aqueous solution of cAP-AGA at 5 mg/ml (in Milli-Q water) and an organic solution of PFP (5 μL/mL). The TFR was set at 10 ml/min and the FRR was set at 3-1

At the end of the microfluidic process, the Samples 7A and 7B were divided into two aliquots as described in previous Example 2 before the dialysis step: the first aliquots (Sample 7A_{ND} and Sample 7B_{ND}) were maintained as such. The second aliquots Sample 7A_{D} and 7B_{D} and the samples 8A, 8B and 8C were diluted 5 times in 2.5% aqueous DMSO. All seven samples were then transferred to a Slide-A-Lyzer™ Dialysis Cassettes from Thermo Fisher Scientific and dialyzed against 2.5% aqueous DMSO overnight with air bubbles for the first hour. Subsequently, sample 7A and sample 7B were further dialyzed against Milli-Q® water for 2 hours and against aqueous glucose 5% solution for 4 hours.

At the end of the dialysis steps, the sizes and PDI of the cross-linked vesicles suspensions were directly measured by DLS.

### Results

TABLE 6
Effect of the cAP/PFC molar ratio on the
size and PDI of the cross-linked vesicles

| Sample | Nr | Dilution | Mean diameter [nm] | PDI |
|---|---|---|---|---|
| 7A_{ND} | 0.011 | No | 700 | 0.39 |
| 7B_{ND} | 0.055 | No | 604 | 0.15 |
| 7A_{D} | 0.011 | Yes | 321 | 0.121 |
| 7B_{D} | 0.055 | Yes | 288 | 0.08 |
| 8A | 0.110 | Yes | 344 | 0.05 |
| 8B | 0.220 | Yes | 210 | 0.13 |
| 8C | 0.330 | Yes | 220 | 0.14 |

Table 6 reports the effect of varying the molar ratio between the cross-linkable amphiphilic peptide (CAP) and the perfluorocarbon. As shown, at a low molar ratio value (i.e. Nr=0.011) size and polydispersity of the cross-linked vesicles were relatively higher with respect to vesicles prepared at higher molar ratio value, for the non-diluted preparations (7A_{ND} and 7B_{ND}). On the other hand, for diluted preparations (7A_{D}(corresponding to Samples 2B, 3C and 6D),7B_{D}, 8A, 8B and 8C) the difference in size and size distribution was lower.

### Example 8

### Effect of the Flow Rate Ratio

Aqueous suspensions of calibrated fluorocarbon cross-linked vesicles were prepared through microfluidic method as described in Example 1. In order to investigate the effect of the Flow Rate Ratio (FRR) on the sizes and the PDI of the cross-linked vesicles, three different samples were prepared:
Sample 8A obtained co-injecting into the microfluidic cartridge at an FRR of 1-1 and a TFR of 10 mL/min an aqueous solution of cAP-AGA (SEQ ID N: 3) at 5 mg/ml (in Milli-Q water) and an organic solution of PFP at 5 μL/mL (in EtOH at 4° C.);
Sample 8B obtained co-injecting into the microfluidic cartridge at an FRR of 2-1 and a TFR of 10 mL/min an aqueous solution of cAP-AGA (SEQ ID N: 3) at 5 mg/ml (in Milli-Q water) and an organic solution of PFP at 5 μL/mL (in EtOH at 4° C.);
Sample 8C, obtained co-injecting into the microfluidic cartridge at an FRR of 3-1 and a TFR of 10 mL/min an aqueous solution of cAP-AGA (SEQ ID N: 3) at 5 mg/ml (in Milli-Q water) and an organic solution of PFP at 5 μL/mL (in EtOH at 4° C.).

At the end of the microfluidic process, all the samples were diluted 5 times in 2.5% aqueous DMSO, then transferred to a Slide-A-Lyzer™ Dialysis Cassettes from Thermo Fisher Scientific and dialyzed against 2.5% aqueous DMSO overnight with air bubbles for the first hour. Subsequently, the samples were further dialyzed against Milli-Q® water for 2 hours (2 times).

At the end of the dialysis steps, the sizes and PDI of the cross-linked vesicles suspensions were directly measured by DLS.

### Results:

TABLE 7
Effect of FRR on the size and PDI of the cross-linked vesicles

| Sample | FRR | Mean diameter [nm] | PDI |
|---|---|---|---|
| 8A | 1-1 | 344 | 0.05 |
| 8B | 2-1 | 210 | 0.13 |
| 8C | 3-1 | 220 | 0.14 |

Table 7 shows the effect of the FRR parameter on the size and PDI values of the cross-linked vesicles obtained through the microfluidic process. As illustrated, increasing the amount of aqueous phase, in comparison with the organic phase, resulted in lower size values while monodispersity remains acceptable and lower than 0.15.

### Example 9

### Effect of the Washing Step

An aqueous suspension of calibrated fluorocarbon cross-linked vesicles was prepared through microfluidic method as described in Example 1. The cross-linkable amphiphilic peptide of formula IV (CAP-AGA; SEQ ID N: 3) was used at a concentration of 5 mg/ml in the aqueous phase and perfluoropentane (PFP) at 10 μL/mL was used in the organic phase. The molar ratio between the cAP-AGA and PFP was 0.055. The TFR was set at 10 ml/min and the FRR was set at 1-1. After the collection from the microfluidic cartridge, the sample was then transferred to a Slide-A-Lyzer™ Dialysis Cassettes from Thermo Fisher Scientific and dialyzed against 2.5% aqueous DMSO overnight with air bubbles for the first hour. Subsequently, the sample further dialyzed against Milli-Q® water for 2 hours and against aqueous glucose 5% solution for 4 hours.

In order to evaluate the effect of the washing step on the size and PDI values of the cross-linked vesicles, at the end of the dialysis process, the obtained suspension was divided into two aliquots: the first aliquot (Sample 5A) was maintained as such and the second one (Sample 9B) was centrifugated for 5 minutes at 4° C. with a rotation of 4000 g. Then the supernatant phase comprising fluorocarbon-free assemblies and residue compounds was separated from the precipitate and discarded.

The centrifugation step was repeated three times.

The precipitate was finally re-suspended adding an aqueous solution of glucose at 5% to obtain an aqueous suspension of calibrated fluorocarbon cross-linked vesicles.

At the end of the cross-linking step, the freshly prepared aqueous suspension of calibrated fluorocarbon cross-linked vesicles

Results

TABLE 8
Effect of the washing step on size and PDI

| Sample | Washing | Mean diameter [nm] | PDI |
|---|---|---|---|
| 5A | No | 588 | 0.19 |
| 9B | Yes | 331 | 0.10 |

As displayed in Table 8, the washing procedure led to a reduction of the PDI value, giving an improved monodispersity.

### Example 10

### Determination of the Cross-Linking Degree.

The determination of the cross-linking degree was performed on a sample of microfluidically-obtained calibrated perfluocarbon cross-linked vesicles having a molar ratio between cAP-AGA (SEQ ID N: 3) and PFP of 0.055 (cAP dissolved water). The percentage of crosslinking was measured by Ellman's assay. Briefly, a 10 mM solution of Ellman's reagent was prepared in 1 mM EDTA, 200 mM phosphate buffer pH=7.5. Two microliters (2 μL) of concentrated suspension or 10 μL of diluted suspension and 4 μL of a solution containing Ellman's reagent were added to 140 μL of PBS buffer. The mixture was incubated in the dark for 40 min. Absorbance was measured and the concentration was calculated using Beer-Lambert law (Eq. 2):

$$A = ε \cdot \vert \cdot C \qquad ( Eq . 2 )$$

wherein A is the absorbance, I is the sample chamber length (cm), ε is the molar absorption coefficient (M⁻¹ cm⁻¹) and C is the concentration.

In this specific example & was 14150 M⁻¹ cm⁻¹.

Four microliters (4 μL) of a solution containing Ellman's reagent in 140 μL of PBS buffer were used as blank.

### Results

The degree of cross-linking was found to be higher than 90%, demonstrating that a substantially high number of cross-linkable cysteine residues comprised in the outer shell of the cross-linked vesicles were intermolecularly connected via disulfide cross-linking groups (-S-S).

SEQUENCE LISTING

```
SEQ ID NO: 1
<210> 1
<211> 7
<212> PRT
<213> Artificial Sequence
<220>
<223> cross-linking motif
<400> 1
Gly Gly Gly Cys Cys Gly Gly
1 5
SEQ ID NO: 2
<210> 2
<211>
<212> PRT
<213> Artificial Sequence
<220>
<223> amphiphilic peptide
<220>
<221> MISC FEATURE
<222> (1) . . . (3)
<223> Each is pentafluoro phenylalanine
(2,3,4,5,6-pentafluoro-L-phenylalanine)
<400> 2
Xaa Xaa Xaa Gly Gly Gly Cys Cys Gly Gly Lys Gly
1 5 10
Ala Gly Ala
                15
SEQ ID NO: 3
<210> 3
<211>
<212> PRT
<213> Artificial Sequence
<220>
<223> amphiphilic peptide
<220>
<221> MISC FEATURE
<222> (1) . . . (3)
<223> Each X is pentafluoro phenylalanine
(2,3,4,5,6-pentafluoro-L-phenylalanine)
<220>
<221> MOD_RES
<222> (15) . . . (15)
<223> AMIDATION
<400> 3
Gly Gly
Xaa Xaa Xaa Gly Gly Gly Cys Cys Gly Gly Lys Gly
1 5 10
Ala Gly Ala
                15
```

### REFERENCES

Melich, R. et al, International Journal of Pharmaceutics, 2020, Volume 587, 119651 WO2019/023706
Sheeran, P. S. et al, Langmuir. 2011, 27 (17): 10412-10420.

1. A method for the preparation of an aqueous suspension of calibrated fluorocarbon cross-linked vesicles, said method comprising the steps of:
a) Preparing an aqueous phase wherein the temperature of said aqueous phase is at least of 10° C.;
b) Preparing an organic phase, wherein
i) said aqueous phase comprises a cross-linkable amphiphilic peptide and said organic phase comprises a fluorocarbon or
ii) said organic phase comprises a cross-linkable amphiphilic peptide and a fluorocarbon;
c) Injecting said aqueous phase in a first inlet and said organic phase in a second inlet of a microfluidic cartridge, thereby mixing said aqueous phase and said organic phase in a mixing portion of the microfluidic cartridge to obtain an aqueous suspension of calibrated fluorocarbon cross-linkable vesicles;
d) Collecting the aqueous suspension of calibrated fluorocarbon cross-linkable vesicles from an exit channel of the microfluidic cartridge, and
e) Cross-linking the cross-linkable amphiphilic peptides to obtain an aqueous suspension of calibrated fluorocarbon cross-linked vesicles.

2. The method according to claim 1, wherein said aqueous phase comprises a cross-linkable amphiphilic peptide surfactant and said organic phase comprises a fluorocarbon.

3. The method according to claim 1, wherein said fluorocarbon is a perfluorocarbon.

4. The method according to claim 1, wherein said aqueous phase comprises an aqueous liquid component having a pH lower than 7.0.

5. The method according to claim 1, wherein said aqueous phase comprises an aqueous liquid component selected from water, aqueous buffered solutions, aqueous isotonic solutions or a mixture thereof.

6. The method according to claim 5, wherein said aqueous liquid component is selected from water, sodium acetate buffer and tris(hydroxymethyl)aminomethane buffer.

7. The method according to claim 1, wherein the temperature of said aqueous phase is at least of 15° C.

8. The method according to claim 7, wherein the temperature of said aqueous phase is at least 20° C.

9. The method according to claim 1 any of the preceding claims, wherein step e) comprises contacting said aqueous suspension of calibrated fluorocarbon cross-linkable vesicles with an oxidizing source able to induce the cross-linking of the cross-linkable amphiphilic peptides to obtain an aqueous suspension of calibrated fluorocarbon cross-linked vesicles.

10. The method according to claim 9, wherein said oxidizing source is contacted with said aqueous suspension of calibrated fluorocarbon cross-linked vesicles for a time sufficient to induce a degree of cross-linking of the amphiphilic peptides of 90% or greater.

11. The method according to claim 9, wherein said oxidizing source comprises a mixture of an oxidizing solution and an oxidizing gas.

12. The method according to claim 9, wherein the step e) is a dialysis procedure.

13. The method according to claim 1, further comprising step d′), which comprises diluting the collected aqueous suspension of calibrated fluorocarbon cross-linkable vesicles.

14. The method according to claim 1, further comprising step f) which comprises washing the obtained aqueous suspension of calibrated fluorocarbon cross-linked vesicles.

15. An aqueous suspension comprising a plurality of calibrated fluorocarbon cross-linked vesicles obtainable by the process as defined in claim 1, wherein said cross-linked vesicles have a z-average diameter comprised between 100 nm and 1000 nm and a polydispersity lower than 0.25.

16. The aqueous suspension according to claim 15, wherein said cross-linked vesicles have a degree of cross-linking higher than 80%.

17. A method of diagnostic treatment of an animal comprising administering the aqueous suspension according to claim to the animal and imaging a part of the animal.

18. A method of therapeutic treatment of a patient comprising administering the aqueous suspension according to claim 15 to the patient and applying an ultrasound to the patient.

19. The aqueous suspension according to claim 15, wherein said cross-linked vesicles have a degree of cross-linking of at least 85%.

20. The aqueous suspension according to claim 15, wherein said cross-linked vesicles have a degree of cross-linking of at least 90%.


SEQUENCE LISTING (4 sequences)

SEQ ID NO: 1 - protein, 7 aa, synthetic construct
  REGION 1..7: note = cross-linking motif
GGGCCGG

SEQ ID NO: 2 - protein, 15 aa, synthetic construct
  REGION 1..15: note = amphiphilic peptide
  SITE 1..3: note = MISC_FEATURE - Each X is pentafluoro phenylalanine (2,3,4,5,6-pentafluoro-L-phenylalanine)
XXXGGGCCGGKGAGA

SEQ ID NO: 3 - protein, 15 aa, synthetic construct
  REGION 1..15: note = amphiphilic peptide
  SITE 1..3: note = MISC_FEATURE - Each X is pentafluoro phenylalanine (2,3,4,5,6-pentafluoro-L-phenylalanine)
  MOD_RES 15: note = AMIDATION
XXXGGGCCGGKGAGA

SEQ ID NO: 4 - protein, 15 aa, synthetic construct
  SITE 1..3: note = Pentafluoro phenylalanine (2,3,4,5,6-pentafluoro-L-phenylalanine)
  VARIANT 15: note = May be amidated
XXXGGGCCGGKGAGA